(19) United States
(12) Patent Application Publication
(10) Pub. No.: US 2008/0138793 A1
(43) Pub. Date: Jun. 12, 2008

(54) Method for cholesterol determination

(75) Inventors: Stellan Lindberg, Forslov (SE); Elisabeth Burestedt, Vejbystrand (SE); Pia Nilsson, Helsingborg (SE)

Correspondence Address: BUCHANAN, INGERSOLL & ROONEY PC, POST OFFICE BOX 1404, ALEXANDRIA, VA, 22313-1404, US

(73) Assignee: HemoCue AB, Angelholm (SE)

(21) Appl. No.: 11/717,684

(22) Filed: Mar. 14, 2007

(30) Foreign Application Priority Data
Dec. 6, 2006 (SE) 0602607-4

(51) Int. Cl. G01N 33/00; A61B 19/00; C12M 1/00

(52) U.S. Cl. 435/4; 435/287.1; 604/403

(57) ABSTRACT

The present invention relates to a sampling device for taking up a blood sample and for providing the blood sample for analysis of total cholesterol in said blood sample. The device comprises a receiving cavity for receiving, through capillary action, the blood sample to be analysed, said receiving cavity having a predetermined small volume; and an analysis cavity, arranged in communication with the receiving cavity, said analysis cavity having a predetermined optical path length. The receiving cavity contains a dried buffer, and the analysis cavity contains a dried reagent. The invention also relates to a method for quantitative determination of the total cholesterol concentration in a blood sample of serum/plasma by end point analysis.

### FIELD OF THE INVENTION

The present invention relates to a device and method for determining total cholesterol in blood. Particularly the invention concerns a sampling device and a method for spectrophotometric measurement of total cholesterol in blood.

### BACKGROUND OF THE INVENTION

Cholesterol is a sterol lipid essential to the cells of the body and mainly produced by the liver. As cholesterol is hydrophobic it cannot be dissolved and transported in the bloodstream directly, but is transported as part of lipoproteins. Most of the blood cholesterol, about 80%, is present as part of LDL (low density lipoprotein) particles, but also other lipoproteins, such as HDL (high density lipoprotein), transport cholesterol. For clinical analytical purposes, both the levels of the LDL and HDL cholesterol are of marked interest, but also the total blood cholesterol concentration is very important.

In connection with the introduction of several new drugs such as statins, for hypercholesterolemia treatment during the end of 1990's, the need for point of care methods, both for screening and monitoring of cholesterol has increased and the clinical demands for precision and accuracy are high with a desirable imprecision around 3%. It is evident that a simple, fast and temperature independent test for quantitative determination of total cholesterol in blood would be an important aid at doctor's offices.

Both chemical and enzymatic methods for total cholesterol measurement are known and measuring of total cholesterol in plasma or serum is performed in central laboratories in hospitals. The most used chemical method is the Liebermann-Burchard reaction, wherein cholesterol reacts as a typical alcohol with strong, concentrated acids producing a coloured substance. Today however mostly enzymatic methods are used. The enzymatic reactions start with a hydrolysis of cholesterol esters to form free cholesterol and the free cholesterol is the oxidized by the enzyme cholesterol oxidase. This enzyme is distinguished by good stability, it is easy to use and it is commercially available.

Another enzyme used in cholesterol determinations is cholesterol dehydrogenase the use of which is disclosed in an analytical element in the patent publication E 0 244 825. According to this publication the sample has to be incubated at a specific temperature for a prescribed time. The use of cholesterol dehydrogenase for determination of cholesterol is also disclosed in e.g. the U.S. Pat. Nos. 4,892,916 and 4,181,575. Both patents concern the determination of total cholesterol by wet chemical methods including long incubation times and defined temperatures.

In order to provide a new, fast and simple method for the determination of total cholesterol in blood a disposable device (microcuvette) including a dry reagent of the type first disclosed in the U.S. Pat. No. 4,088,448 was especially studied as the use of this type of microcuvette offers several advantages. This microcuvette permits sampling of a liquid, mixing the sample with a suitable reagent, for instance for colour development, in the same vessel as the one used for the subsequent measurement. Furthermore the sampling procedure is simplified, the number of utensils is reduced and in most cases, depending upon the type of analysis, the exactitude of the analysis is considerably improved by making the analysing procedure independent of the operating technique of the operator making the analysis. The procedure is also remarkably fast as it permits the liquid sample to be instantly mixed with the reagent and then permits measurement shortly afterwards, without time consuming intermediary steps.

### OBJECTIVES OF THE INVENTION

An objective of the invention concerns a sampling device and a method for simple quantitative determination of total cholesterol in blood.

An other objective of the invention concerns a sampling device and a method for quantitative determination of total cholesterol in blood which method can be performed at ambient temperature.

An other objective of the invention is to provide a sampling device including a cholesterol reagent composition which can be stored for prolonged periods of time.

Still an other objective of the invention is to provide a sampling device for quantitative rapid determination of total cholesterol in blood.

Still an other objective of the invention is to provide a sampling device for quantitative end-point determination of total cholesterol in blood.

Still an other objective of the invention is to provide a sampling device for quantitative determination of total cholesterol in serum/plasma, wherein undiluted whole blood is introduced into the device.

### SUMMARY OF THE INVENTION

In developing the inventive sampling device and method several attempts to use the cholesterol oxidase in a microcuvette in accordance with the U.S. Pat. No. 4,088,448 (as discussed above) failed and no reproducible results could be obtained. It is believed that these failures were due to the fact that the construction of the microcuvette with the capillary opening creates a specific reaction environment in the microcuvette and that this environment is not suitable for reactions with cholesterol oxidase.

Nor did the other enzyme, cholesterol dehydrogenase, work satisfactorily in combination with the other reagents necessary for the cholesterol determination. However, it was unexpectedly found that if a buffering agent, needed for the reaction, was separated from the rest of the reagent composition including the dehydrogenase in the device, a successful temperature independent end-point quantitative determination of total cholesterol could be obtained.

It was thus found that the objectives above were achieved fully or partly through an inventive sampling device and also through employing an inventive method, as defined below.

The inventive sampling device is a sampling device for taking up a blood sample and for providing the blood sample for analysis of total cholesterol in said blood sample, said device comprising: a receiving cavity for receiving, through capillary action, the blood sample to be analysed, said receiving cavity having a predetermined small volume; and an analysis cavity, arranged in communication with the receiving cavity said analysis cavity having a predetermined optical path length; said receiving cavity containing a dried buffer, and said analysis cavity containing a dried reagent.

The inventive method is a method for quantitative determination of the total cholesterol concentration in blood sample of serum/plasma by end point analysis comprising: a) contacting serum/plasma with a dried buffer, whereby the buffer is dissolved in the blood sample, buffering the same; b) contacting a small, defined volume of the buffered serum/plasma with a dried reagent, said reagent comprising cholesterol dehydrogenase; cholesterol esterase; one or more substances from the group consisting of diaphorase, phenazine methosulphate, phenazine ethosulphate, phenazine phenosulphate and Meldola blue; one or more substances from the group consisting of NAD, NADP, thio-NAD, thio-NADP, nicotin-amide-purine dinucleotide, nicotinamide-methylpurine dinucleotide and nicotinamide-2-chloro-methylpurine dinucleotide; one or more surfactants from the group consisting of polyoxyethylenes, alkyl glucosides, thioglucosides, copolymer and bile acids; and a redox indicator dye; and c) measuring by transmission spectrophotometry, over a predetermined optical path length, the colour change brought about by the reaction of the reagent with cholesterol in the defined volume of the buffered serum/plasma to quantitatively determine the cholesterol concentration therein.

### BRIEF DESCRIPTION OF THE DRAWINGS

FIG. 1 is a schematic front view of one embodiment of the inventive sampling device. FIG. 1 also schematically illustrates one embodiment of the use of said sampling device.

FIG. 2 is a graph disclosing the correlation between cholesterol determination according to an embodiment of the inventive method and a reference method.

### DETAILED DESCRIPTION OF THE INVENTION

The invention relates in particular to total cholesterol determination in small volumes of blood. In this context the term “blood” is intended to mean whole blood, plasma and/or serum. According to a preferred embodiment of the invention, the blood which is introduced into the device is neither diluted nor pretreated. The term “small volumes” means volumes between 0.1 and 0.001 ml, preferably between 0.06 and 0.001 ml.

The designation “serum/plasma” or “plasma/serum” is intended to encompass blood serum, blood plasma and any intermediate stages in between. The reason for sometimes not explicitly defining a blood fraction as “serum” or “plasma” is that if a blood sample is analysed, without addition of anti-coagulants, within a few minutes after acquiring the blood from a patient, true serum will not have time to form, and the measurement is made on an intermediary stage between plasma and serum after removal of the blood cells. If the removal of cells and the measurement is made almost directly after acquiring the blood from a patient, the measurement will essentially be made on plasma. A blood component which is present in both plasma and serum will, due to the removal of the fibrinogen from serum, be present in a concentration of 3% less in plasma as compared with the concentration in serum.

In this application, the term “cavity” is intended to be construed as a volume or chamber defined by wall surfaces. The cavities (volumes) of the device according to the present invention are not however completely, defined, or enclosed, by these surfaces, but have inlets and/or outlets where the surfaces are not completely joined together. The cavities (volumes) are usually not evacuated, but may contain a gas (generally air), a dried reagent and/or buffer, a liquid (such as a blood sample when the device is in use), etc.

The sampling device of the present invention is designed in such a way that it holds the dried buffer separate from the dried reagent, thus making it possible to keep the reagent at a pH irrespective of the buffer pH while the reagent is in dried form (e.g. during storage of the sampling device). By being able to optimise the reagent pH for storage stability, regardless of the optimum reagent pH for reacting with a blood sample, the shelf life of the inventive device can be greatly improved. This is achieved by equipping the sampling device with a plurality of cavities.

In other words, if a blood sample is first contacted with the buffer, dissolving it, and then contacted with the reagent, dissolving it too, the compounds of the reagent are only subjected to the pH as invoked by the buffer after being contacted with the blood sample. As the dried reagent is only contacted with the sample at the time it is intended to react with the same, problems with long term stability of compounds of the reagent at the buffer invoked pH are avoided with this sampling device.

In its simplest form, a serum/plasma sample is introduced into the sampling device, and in this case only the receiving and analysis cavities are needed. However, in its commercially most interesting form, a sample of undiluted whole blood is introduced into the device. In this case the device has to be designed so as to include additional cavities adapted for removal of the blood cells of the sample through centrifugation, as the cells, if present in the analysis cavity, will interfere with the cholesterol determination. A device which discloses the removal of blood cells by centrifugal action and which may be used is e.g. the device of the U.S. Pat. No. 5,472,671 (which is hereby incorporated by reference).

The receiving cavity has a predetermined volume, which allows it to receive a predetermined, invariable volume of blood (serum/plasma) which may then be transferred to the analysis cavity. This ensures that a specific and known volume is reacted with the dried reagent in the analysis cavity.

The analysis cavity has a predetermined optical path length, which ensures that when a photometer is used over the analysis cavity for obtaining a measurement value, this value may be directly correlated to the cholesterol concentration of a blood sample therein.

For the determination of total cholesterol in a serum fraction the inventive method may thus also include centrifugation of whole blood for removing blood cells and fibrinogen from the whole blood before the serum thus obtained is contacted with the dry reagent in the sampling device.

Similarly, for the determination of total cholesterol in a plasma fraction, the inventive method may include contacting unaltered whole blood with an anti-coagulating agent and subjecting the obtained mixture to centrifugation for removing blood cells before the plasma thus obtained is contacted with the dry reagent in the sampling device.

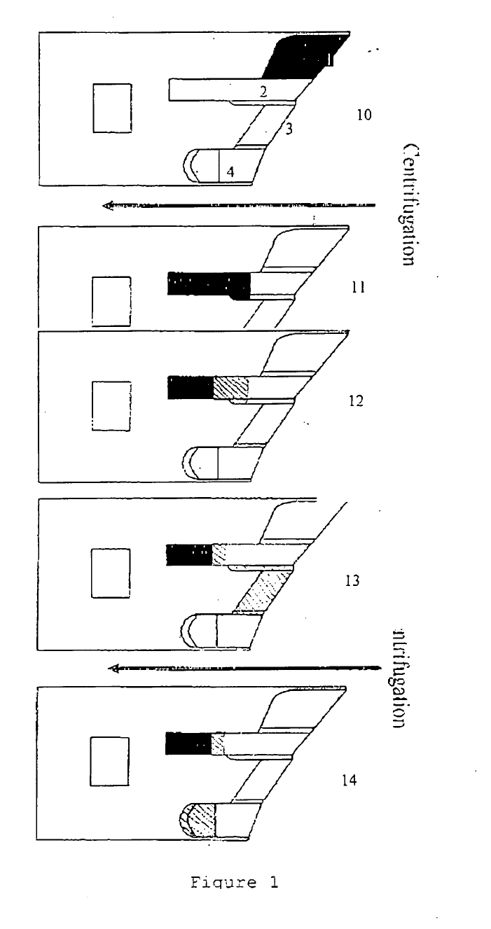

These preparatory steps, centrifugation optionally preceded by use of an anti-coagulating agent, may be performed before the blood sample is introduced into the sampling device, or after introduction of whole blood through e.g. centrifugation of the sampling device. In the latter case, with reference to FIG. 1, the use of a sampling device comprising four cavities may be employed: a first cavity (1) (an inlet cavity) in connection with the surroundings of the sampling device via an inlet, and optionally containing a dry additive such as a wetting agent, for receiving a sample of blood, preferably whole blood, from outside of the device through capillary action; a second cavity (2) (a centrifugation reception cavity), preferably non-capillary, connected to the first cavity (1) and into which the sample may be transferred through centrifugal action; a third cavity (3) (the receiving cavity), preferably capillary and containing the dried buffer and optionally a wetting agent, in connection with the second cavity (2) and into which the serum/plasma fraction of the sample ray be transferred through capillary action upon end of centrifugation; and a fourth cavity (4) (the analysis cavity), connected to the third cavity (3) and into which the sample may be transferred through centrifugal action, containing the dried reagent.

A preferred use of the four cavity device above is to introduce whole blood into the device directly from a pricked finger of a patient. With reference to FIG. 1, the blood is first (10) drain into the inlet cavity (1) through capillary action. This may be aided by a wetting agent in dry form deposited in the inlet cavity (1) at manufacture of the device, and by the device having a pointy design providing a point at the inlet of the inlet cavity (1) which may make contact with the blood of the pricked finger. The device may then be subjected to centrifugal action (11), such that the blood is transferred from the inlet cavity (1) to the centrifugation reception cavity (2), and such that the blood cells of the blood are essentially separated from the plasma (12) Upon end of centrifugation the blood plasma is, through capillary action, drawn from the centrifugation reception cavity (2) into the receiving cavity (3), where dried buffer is quickly dissolved in a specific volume of the plasma defined by the volume of the receiving cavity (3) (13). After the buffer has dissolved in the plasma, thus buffering the same, the device is again subjected to centrifugation, whereby the buffered plasma is transferred to the analysis cavity (4) where the dried reagent for the cholesterol determination is dissolved in the buffered plasma (14). After reaction with the reagent the total cholesterol of the plasma is determined through absorption photometry.

The sampling device may be disposable, i.e. it is arranged to be used only once. The sampling device provides a kit, which can be stably stored for a long time before use, for performing a determination of total cholesterol, since the sampling device is able to receive a liquid sample and holds all reagents needed in order to present the sample to cholesterol measurement. This is particularly enabled if the sampling device is adapted for use only once and may be formed without consideration of possibilities to clean the sampling device and re-apply a reagent. Identical units of the inventive sampling device may be mass produced with a very low tolerance for deviations, whereby measurements made using one specific unit may be directly compared with measurements made using other units of the same inventive sampling device.

Also, the sampling device may be moulded in a plastic material and thereby be manufactured at a low cost. Thus, it may still be cost-effective to use a disposable sampling device.

Further, by forming the sampling device from a rigid elastic material, the devise may not be deformed during handling and use of the device, thus ensuring invariable volumes and shapes of the device cavities after manufacture, consequently also ensuring an invariable optical path length.

According to the embodiment of the inventive method the following reaction steps are performed with the indicated reagent ingredients:

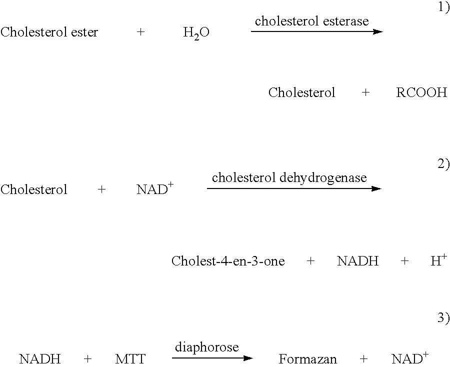

The ingredients of the dried reagent are not restricted to those exemplified in the above reaction scheme, but are discussed in some detail below.

The cholesterol esterase may be obtained from different species having different molar weights, pH optima etc.

The coenzyme may be NAD, preferably β-NAD, NADP, thio-NAD, thio-NADP, nicotinamide-purine dinucleotide, nicotinamide-methylpurine dinucleotide and nicotinamide-2-chloro-methylpurine dinucleotide.

Also cholesterol dehydrogenase can be obtained from different species having different molar weights, pH optima etc. Examples of publications concerning cholesterol dehydrogenase are the Japanese Patents Laid-open Nos. 89,183/1983 and 89,200/1983, wherein the preparation of cholesterol dehydrogenase is disclosed. In the inventive method cholesterol dehydrogenase only encompasses NAD- or NAD-analog-dependent enzymes.

Diaphorase can also be obtained from different species and is commercially available. Diaphorase can however be replaced by known substances, such as phenazine methosulphate, phenazine ethosulphate, phenazine phenosulphate, Meldola blue etc. There are also other known NAD-analogs, such as the best known NADP, which can be reduced by the cholesterol dehydrogenase reaction and transfer the reduction to a dye or colour system.

MTT (3-(4,5-dimethylthiazole-2-1)-2,5-diphenyl-2H-tetrazolium bromide) is an example of a redox indicator dye, which yields a good result when used in the inventive method, although many other tetrazolium compounds can be used. There are also several other known types of colour-changing substances, which are capable of changing colour when affected by NADH and diaphorase. Tetrazolium compounds are advantageous in that the formazan dye is formed irreversibly under normal reaction conditions. According to a preferred embodiment MTT is used as redox indicator dye and the absorbance is measured in the range 630-680 nm, most specifically 640 nm with a measurement for background correction in the rang 700-900 nm or more specifically at 700 nm or 840 nm. The wavelength for the absorbance measurement depends on the redox dye used. For dyes suitable to be used according to the inventive method the wave length may vary between 500 and 750 nm.

In addition, the reagent can contain non-ionic surfactants such as polyoxyethylenes and/or alkyl glucosides and/or thio-glucosides and/or copolymer and/or anionic surfactants such as bile acids or enzyme such as phospholipas as agents for lysing the lipoprotein. In addition surfactants may be used for wetting the dry reagent matrix. Ideally, the surfactant(s) should exhibit the following characteristics:
1. cause rapid setting of the dry reagent by reducing the surface tension.
2. lyse the lipoprotein to release the cholesterol and cholesterol ester
3. keep the formed formazan solubilized

The contents of the different components in the dried reagent composition are not critical, but calculated on a sample of 1 ml undiluted whole blood may preferably be in the following ranges:

| Substance | Quantity | |
|---|---|---|
| Cholesterol esterase | 20–5000 | U/mL |
| Cholesterol dehydrogenase | 20–5000 | U/mL |
| Diaphorase(/analogs) | 20–50000 | U/mL |
| β-NAD (/analogs) | 12–100 | mmol/mL |
| Redox indicator dye/ | 12–25 | mmol/mL |
| Tetrazolium salt (MTT-Br) | | |
| (/analogs) | | |
| Triton X-100 | 0.1–5% | (w/w) |

The above substances are mixed in order to form a suspension, which may be freeze-dried in the analysis cavity of the inventive sample device.

The invention is illustrated by the following non-limiting example.

### EXAMPLE

### Determination of Cholesterol in Serum/Plasma.

A reagent solution including 1% triton X-100 in water was prepared. MTT (3-(4,5-dimethylthiazole-2-1)-2,5-diphenyl-2H-tetrazolium bromide) was added to the solution and mixed until the MTT was dissolved. An aqueous solution of cholesterol dehydrogenase, cholesterol esterase, diaphorase and β-NAD was added to the obtained solution and 3-6 μl of this solution were filled into the analysis cavity of the inventive disposable sampling devices.

1 ml of the reagent composition included:

200 U Cholesterol esterase, Genzyme

200 U Cholesterol dehydrogenase, Amano

1700 U Diaphorase, Unitika

20 mM β-NAD, Sigma

15 mM HTT-Br, Acros

10 mg Triton X-100, Merck

1 mL water subjected to ion-exchange

A Tris buffer solution having a pH of 9.0 was in a similar way filled into the receiving cavity of the inventive disposable sampling devices.

The sampling devices including the reagent and buffer were frozen at −45° C. and freeze-dried in order to obtain sampling devices including a dried reagent and a dried buffer in respective cavities.

One obtained sampling device was used as follows:

A defined serum/plasma sample volume is drawn into the receiving cavity by capillary action. The dried buffer dissolves in the serum/plasma. The sampling device is then subjected to centrifugation such that the buffered serum/plasma is forced into the analysis cavity containing the dried reagent. The dried reagent composition dissolves in the serum/plasma, whereby the pH changes to 8.5, and the serum/plasma cholesterol is reacted with cholesterol esterase and cholesterol dehydrogenase as defined in the above chemical reactions. The chemical reactions lead to a dye concentration change. By transmission spectrometry measurements at 640 nm and compensation for background at 840 nm the concentration of cholesterol in the serum/plasma sample can be quantitatively determined. The whole process, from drawing in the sample to the measurement, typically takes less than 5 minutes to perform, usually about 2 minutes.

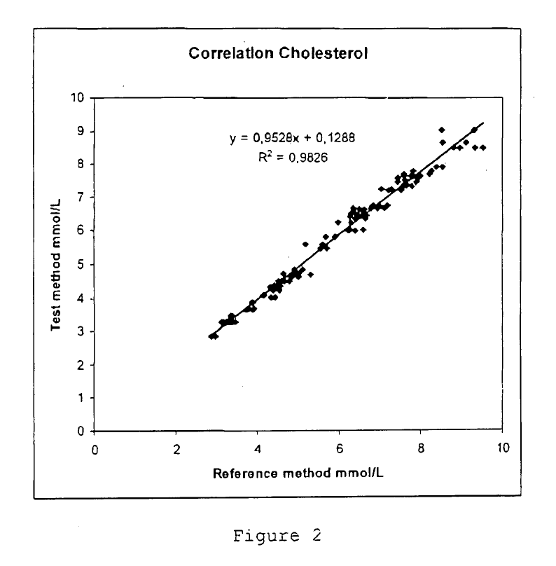

FIG. 2 discloses the relationship between the cholesterol determination according to the present invention and a reference method for end-point determination, and as can be seen the agreement is good.

1. A sampling device for taking up a blood sample and for providing the blood sample for analysis of total cholesterol in said blood sample, said device comprising:
a receiving cavity for receiving, through capillary action, the blood sample to be analysed, said receiving cavity having a predetermined small volume; and
an analysis cavity, arranged in communication with the receiving cavity, said analysis cavity having a predetermined optical path length;
said receiving cavity containing a dried buffer, and said analysis cavity containing a dried reagent.

2. The device according to claim 1, wherein the device is manufactured from a rigid material and the analysis cavity has an invariable optical path length.

3. The device according to claim 1, wherein the buffer contained in the receiving cavity has a pH of 8-10.

4. The device according to claim 1, wherein the buffer contained in the receiving cavity has a pH of about 9.

5. The device according to claim 1, wherein the receiving cavity contains a wetting agent.

6. The device according to claim 1, wherein the analysis cavity is in communication with the receiving cavity such that spontaneous flow of the body fluid from the receiving cavity to the analysis cavity is prevented and such that the blood of the blood sample may be forced from the receiving cavity to the analysis cavity by applying centrifugal action to the device.

7. The device according to claim 1, wherein the reagent contained in the analysis cavity comprises
cholesterol dehydrogenase;
cholesterol esterase;
one or more substances from the group consisting of diaphorase, phenazine methosulphate, phenazine ethosulphate, phenazine phenosulphate and Meldola blue;
one or more substances from the group consisting of NAD, NADP, thio-NAD, thio-NADP, nicotinamide-purine dinucleotide, nicotinamide-methylpurine dinucleotide and nicotinamide-2-chloro-methylpurine dinucleotide;
one or more surfactants from the group consisting of polyoxyethylenes, alkyl glucosides, thio-glucosides, copolymer and bile acids; and
a redox indicator dye.

8. The device according to claim 7, wherein the redox indicator dye is 3-(4,5-dimethylthiazole-2-1)-2,5-diphenyl-2H-tetrazolium bromide (MTT).

9. The device according to claim 1, wherein the reagent comprises diaphorase.

10. The device according to claim 1, further comprising
an inlet cavity for taking up blood from a location externally of the device through capillary action; and
a centrifugation reception cavity arranged in communication with the inlet cavity such chat spontaneous flow of the blood from the inlet cavity to the centrifugation reception cavity is prevented and such that the blood may be forced into the centrifugation reception cavity from the inlet cavity by applying centrifugal action to the device,
said centrifugation reception cavity being in capillary connection with the receiving cavity for providing transport of fluid from the centrifugation reception cavity to the receiving cavity by capillary action.

11. A method for quantitative determination of the total cholesterol concentration n a blood sample of serum/plasma by end point analysis comprising:
a) contacting serum/plasma with a dried buffer, whereby the buffer is dissolved in the blood sample, buffering the same;
b) contacting a small, defined volume of the buffered serum/plasma with a dried reagent, said reagent comprising
cholesterol dehydrogenase;
cholesterol esterase;
one or more substances from the group consisting of diaphorase, phenazine methosulphate, phenazine ethosulphate, phenazine phenosulphate and Meldola blue;
one or more substances from the group consisting of NAD, NADP, thio-NAD, thio-NADP, nicotinamide-purine dinucleotide, nicotinamide-methylpurine dinucleotide and nicotinamide-2-chloro-methylpurine dinucleotide;
one or more surfactants from the group consisting of polyoxyethylenes, alkyl glucosides, thio-glucosides, copolymer and bile acids; and
a redox indicator dye; and
c) measuring by transmission spectrophotometry, over a predetermined optical path length, the colour change brought about by the reaction of the reagent with cholesterol in the defined volume of the buffered serum/plasma to quantitatively determine the cholesterol concentration therein.

12. The method according to claim 11, wherein the redox indicator dye is 3-(4,5-dimethylthiazole-2-1)-2,5-diphenyl-2H-tetrazolium bromide (MTT).

13. The method according to claim 11, wherein the transmission spectrophotometry measurement is conducted in the range of 630-680 nm.

14. The method according to claim 11, wherein the transmission spectrophotometry measurement is conducted at about 640 nm.

15. The method according to claim 11, further comprising conducting a second transmission spectrophotometry measurement, to compensate for background interference, at a wavelength above 700 nm.

16. The method according to claim 11, wherein the reagent comprises diaphorase.

17. The method according to claim 11, wherein the small, defined volume of the buffered plasma or serum is between 0.1 and 0.001 ml.

18. The method according to claim 11, wherein the small, defined volume of the buffered plasma or serum is between 0.03 and 0.001 ml.

19. The method according to claim 11, said method further including centrifugation of whole blood for removing the red blood cells from the whole blood before performing step a) for the determination of the cholesterol concentration in the serum/plasma fraction of said whole blood.

20. The method according to claim 11, wherein the measurement is performed at a serum/plasma pH of 8-9.

21. The method according to claim 11, wherein the measurement is performed at a serum/plasma pH of about 8.5.
